(19) United States
(12) Patent Application Publication
(10) Pub. No.: US 2021/0002372 A1
(43) Pub. Date: Jan. 7, 2021

(54) METHODS AND COMPOSITIONS FOR TREATING NATALIZUMAB-ASSOCIATED PROGRESSIVE MULTIFOCAL ENCEPHALOPATHY

(71) Applicants: United States Government as Represented by the Department of Veterans Affairs, Washington, DC (US); University of Texas Southwestern Medical Center, Dallas, TX (US)

(72) Inventors: Olaf Stüve, Dallas, TX (US); Rehana Z. Hussain, Dallas, TX (US); Petra C. Cravens, Dallas, TX (US); Darin Okuda, Dallas, TX (US)

(21) Appl. No.: 16/977,265

(22) PCT Filed: Mar. 1, 2019
(86) PCT No.: PCT/US2019/020380

§ 371 Date: Sep. 1, 2020

Related U.S. Application Data
(60) Provisional application No. 62/637,606, filed on Mar. 2, 2018.

(51) Int. Cl. C07K 16/28; A61K 31/713; A61K 31/785

(52) U.S. Cl. CPC C07K 16/2839; A61K 2039/505; A61K 31/785; A61K 31/713

(57) ABSTRACT

Disclosed herein are methods and compositions useful in natalizumab-associated progressive multifocal encephalopathy.

### CROSS REFERENCE TO RELATED APPLICATIONS

This application claims the benefit of U.S. Provisional Application No. 62/637,606, filed on Mar. 2, 2018, the entire content of which is hereby incorporated by reference.

### STATEMENT REGARDING FEDERALLY FUNDED RESEARCH

This invention was made with government support under grant number I01BX001674 awarded by the United States Department of Veterans Affairs. The government has certain rights in the invention.

### BACKGROUND

Antagonizing α4-integrin with natalizumab reduces the trafficking of immune cells into the central nervous system (CNS) and is effective in ameliorating disease activity in patients with multiple sclerosis (MS). However, approximately 1 in 100 recipients of natalizumab will develop progressive multifocal encephalopathy (PML), a potentially fatal opportunistic infection of the CNS. Diminished CNS immune surveillance, and specifically a reduction in the number of activated T lymphocytes in the brain and spinal cord is thought to contribute to the substantial risk of PML under natalizumab.

### BRIEF DESCRIPTION OF THE DRAWINGS

FIGS. 1A-E show the results of generating and utilizing a model to assess the effects of TLR3 agonism on CNS immune re-constitution in the setting of relative α4-integrin deficiency. FIG. 1A shows that Poly I:C engagement of TLR3 results (FIG. 1B) in the expression of type I interferons, which (FIG. 1C) subsequently bind IFN type I receptors in adjacent IFN Type I receptor-expressing cells. FIG. 1D shows that, consequently, downstream transcription factors translocate to the cell nucleus, and start transcription of antiviral genes, including Mx1. FIG. 1E shows that this model allows the conditional deletion of α4-integrin on IFN type I receptor-expressing cells, which includes leukocytes.

FIGS. 2A-I shows that the frequency of α4-integrin (CD49d)-positive leukocytes is reduced in primary and secondary lymphoid organs of poly I:C treated Mx1.Cre⁺α4-integrin^{fl/fl} mice. FIG. 2A—shows that to ablate α4-integrin, Mx1.Cre⁺α4^{fl/fl} mice received 3 intra peritoneal injections of 300 μg poly(I)-poly(C) (poly I:C; Sigma Chemical Company, St. Louis, Mo.) given at 2 days intervals in order to activate the Cre recombinase. This was followed by a “wash-out” period of three weeks in which mice were then analyzed or immunized for EAE. FIG. 2B shows that EAE disease incidence, onset, clinical severity are similar between Mx1.Cre⁺α4-integrin^{fl/fl} mice and C57BL/6 control mice not exposed to poly I:C In the lymph nodes (FIG. 2C), spleen (FIG. 2D), and bone marrow (FIG. 2E) of poly I:C-treated Mx1.Cre⁺α4-integrin^{fl/fl} mice, the frequency of α4-integrin expressing CD3⁺ T cells, CD8⁺ T cells, CD11c⁺ monocyte-derived dendritic cells (DC), and CD22b⁺ Ly6G⁺ myeloid-derived granulocytes is significantly diminished. In spleen (FIG. 2D), and bone marrow (FIG. 2E), the frequency of α4-integrin expressing CD4⁺ T cells, CD19⁺ B cells, and CD22b⁺ Ly6G⁻ macrophages is also significantly reduced. FIG. 2F shows that the transfer of cells from both strains resulted in the onset of clinic disease at day 7. FIG. 2G shows that there was a significant reduction in the migration of CD45⁺ splenocytes from poly I:C-treated Mx1.Cre⁺α4-integrin^{fl/fl} mice was observed when compared to CD45⁺ splenocytes from poly I:C-treated C57BL/6 mice while no significant difference in the migratory capacity of lymphocyte subsets (FIG. 2H), or (I) myeloid cell subsets (FIG. 2I) was observed. *p<0.05, **p<0.01, ***p<0.001, ****p<0.0001.

FIGS. 3A-H shows that in vivo systemic TLR3 agonism through poly I:C treatment re-establishes EAE disease susceptibility and CNS immune competence in the setting of relative α4-integrin deficiency. FIG. 3A shows that when active EAE was induced in Mx1.Cre⁺α4-integrin^{fl/fl} mice and C57BL/6 control mice that were treated with poly I:C, EAE disease incidence, susceptibility and severity were similar in both groups. No differences in composition of leukocytes in lymph nodes (FIG. 3B), spleen (FIG. 3C), brain (FIG. 3D) and spinal cord nodes (FIG. 3E) between the two strains were observed, indicating a full cellular immune re-constitution. FIG. 3F shows that there was no difference between the capacity of Mx1.Cre⁺α4-integrin^{fl/fl} mice and C57BL/6 control mice that were treated with poly I:C to mount recall responses to MOGₚ₃₅₋₅₅. FIG. 3G shows that the number of activated CD4⁺CD25⁺ T cells was increased in the brain of Mx1.Cre⁺α4-integrin^{fl/fl} mice treated with poly I:C, and similar between both mouse strains in the spinal cord. FIG. 3H shows that in the brain and spinal cord, a significant expansion of CD19⁺SSC^{hi} B cells in Mx1.Cre⁺α4-integrin^{fl/fl} mice treated with poly I:C was observed.

FIGS. 4A-I show that TLR3 agonism through poly I:C treatment results in compromise of the blood-brain barrier. FIG. 4A shows that there was no difference in the amount of EBD detected in the CNS of Mx1.Cre⁺α4-integrin^{fl/fl} mice and C57BL/6 control mice treated in vivo with poly I:C. FIGS. 4B-E show that there was no difference in the absolute number of inflammatory infiltrates in the spinal cords between animals of both mouse strains in whom active EAE had been induced in the absence of presence of poly I:C. The anatomical locations of BBB compromise of Mx1.Cre⁺α4-integrin^{fl/fl} mice and C57BL/6 control mice treated vivo with poly I:C as indicated by EBD extravasation differed between mouse strains in the brains (FIGS. 4F, 4G), and in the spinal cords (FIGS. 4H, 4I).

FIGS. 5A-L show that in vivo TLR3 agonism through systemic poly I:C administration promotes diverse integrin usage in CNS-infiltrating leukocytes in the setting of relative α4-integrin deficiency. The expression of Lymphocyte-function associated antigen-1 (LFA-1; β2-integrin; CD11a; FIGS. 5A-D), α5-integrin (CD49e; FIGS. 5E-H), and α4-integrin (CD49d; FIGS. 5I-L) on different lymphocyte and myeloid cell subsets in Mx1.Cre⁺α4-integrin^{fl/fl} mice and C57BL/6 control mice actively induced for EAE on day 15.

FIGS. 6A-B show that systemic TLR3 agonism through poly I:C differentially impacts cytokine expression in a compartment-specific manner in the setting of relative α4-integrin deficiency. FIG. 6A shows a decreased transcription of IFNβ in the brain and the upregulation of several pro-inflammatory cytokines. FIG. 6B shows that the transcription of numerous interleukins, GM-CSF, and the transcription factor FoxP3 was diminished in Mx1.Cre⁺α4-integrin^{fl/fl} mice and in spinal cord, that the transcription of IFNβ in Mx1.Cre⁺α4-integrin^{fl/fl} mice was indistinguishable from that in C57BL/6 control mice. Data is shown as a fold change compared to transcription in C57BL/6 control mice (defined as 1, indicated by a dotted red line).

### SUMMARY

Disclosed herein are methods of treating multiple sclerosis in a subject, the methods comprising: (a) identifying a subject in need of treatment; and (b) administering to the subject a therapeutically effective amount of natalizumab and a toll-like receptor 3 (TLR3) agonist.

Disclosed herein are methods of treating a patient at risk of having progressive multifocal leukoencephalopathy (PML), the methods comprising: administering to a patient a therapeutically effective amount of a toll-like receptor 3 (TLR3) agonist.

Disclosed herein are methods of activating T cells in a subject, the methods comprising: administering a therapeutically effective amount of a composition comprising polyinosinic-polycytidylic acid (Poly (I:C)) to a subject having or suspected of having a reduced number of T cells; wherein the subject has previously undergone treatment with natalizumab or is currently undergoing treatment with natalizumab; and wherein the number of T cells is increased after administration of the Poly (I:C).

Disclosed herein are methods of preventing progressive multifocal leukoencephalopathy (PML) in a subject with multiple sclerosis or Crohn's disease, the methods comprising: (a) identifying a subject in need of treatment; and (b) administering to the subject a therapeutically effective amount of a toll-like receptor 3 agonist before, during or after administration of natalizumab, in an amount sufficient to prevent PML.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for cancer, such as, for example, prior to the administering step. The term “cancer patient” can refer to a subject having a cancer described herein, including a subject diagnosed to suffer from a cancer, but also includes a subject, for example, during or after therapy.

As used herein, the term “comprising” can include the aspects “consisting of” and “consisting essentially of.” “Comprising can also mean “including but not limited to.”

“Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100%, or any amount of reduction in between as compared to native or control levels. In an aspect, the inhibition or reduction is 10-20, 20-30, 30-40, 40-50, 50-60, 60-70, 70-80, 80-90, or 90-100% as compared to native or control levels. In an aspect, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in an aspect, the increase or promotion can be a 10, 20, 30, 40, 50, 60, 70, 80, 90, 100%, or more, or any amount of promotion in between compared to native or control levels. In an aspect, the increase or promotion is 10-20, 20-30, 30-40, 40-50, 50-60, 60-70, 70-80, 80-90, or 90-100% as compared to native or control levels. In an aspect, the increase or promotion is 0-25, 25-50, 50-75, or 75-100%, or more, such as 200, 300, 500, or 1000% more as compared to native or control levels. In an aspect, the increase or promotion can be greater than 100 percent as compared to native or control levels, such as 100, 150, 200, 250, 300, 350, 400, 450, 500% or more as compared to the native or control levels.

As used herein, the terms “disease” or “disorder” or “condition” are used interchangeably referring to any alternation in state of the body or of some of the organs, interrupting or disturbing the performance of the functions and/or causing symptoms such as discomfort, dysfunction, distress, or even death to the person afflicted or those in contact with a person. A disease or disorder or condition can also related to a distemper, ailing, ailment, malady, disorder, sickness, illness, complaint, affection.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, certain changes and modifications may be practiced within the scope of the appended claims.

Antagonism of alpha (α4) beta(β)1-integrin (also: Cluster of differentiation (CD) 49d, very late activation antigen-4; VLA-4) with antibodies substantially reduces leukocyte trafficking into the central nervous system (CNS), and the accumulation of clinical disease activity (Yednock et al., Nature 1992; 356(6364): 63-6; Theien et al., J Clin Invest 2001; 107(8): 995-1006) in the experimental autoimmune encephalomyelitis (EAE) model of the human CNS inflammatory multiple sclerosis (MS) (Stuve and Zamvil, Neurologic diseases. In: Parslow T G, Stites D P, Ten A I, Imboden J B, editors. Medical Immunology. San Francisco: McGraw Hill; 2001. p. 510-26). Based on these findings, a humanized monoclonal IgG₄ antagonist called natalizumab (Tysabri®) was developed for treatment of MS (Shirani and Stuve, J Immunol 2017; 198(4): 1381-6). Natalizumab blocks α4-integrin on the surface of all leukocytes, and consequently diminishes their capability to migrate from the blood into the CNS and the gastrointestinal tract. Following and extensive clinical study program (Miller et al., N Engl J Med 2003; 348(1): 15-23; Polman et al., N Engl J Med 2006; 354(9): 899-910; Rudick et al., N Engl J Med 2006; 354(9): 911-23), natalizumab was approved for patients with relapsing forms of MS in 2005. It is considered one of the most effective therapies for patients with this disorder.

It was been previously demonstrated that continuous natalizumab administration to patients caused a prolonged reduction of lymphocyte subsets present in cerebrospinal fluid (Stuve et al., ArchNeurol 2006; 63(10): 1383-7; Stüve et al., Annals of neurology 2006; 59(5): 743-7; Stuve et al., Neurology 2009; 72(5): 396-401; Kowarik et al., Neurology 2011; 76(14): 1214-21). Another study done by del Pilar Martin et al shows that natalizumab significantly reduced the number of CD4⁺ T cells and dendritic cells (DC) in cerebrovascular spaces (CPVS), an important site of antigen presentation in the brain (del Pilar Martin et al., Archives of neurology 2008; 65(12): 1596-603). These data suggest that natalizumab, through antagonism of α4-integrin, may negatively impact immune surveillance of the CNS.

Three months after the initial approval of natalizumab, Biogen Idec Inc. and Elan Corp voluntarily withdrew natalizumab from the market in February 2005, after three recipients of natalizumab developed multifocal leukoencephalopathy (PML). PML is a rare opportunistic infection of the CNS caused by a human polyomavirus JC (Berger and Houff, Neurological research 2006; 28(3): 299-305). Infection of oligodendrocytes by JC virus (JCV) results in their demise, and demyelination of the brain. PML is most prevalent in the setting of severe and prolonged immunosuppression, and prior to the introduction of natalizumab occurred predominantly in patients with acquired immune deficiency syndrome (AIDS). Despite the identification of modifiable risk factors for PML under natalizumab, the most recent published data suggest that the overall risk of developing PML under natalizumab is approximately 1 in100 recipients (Cutter and Stuve, Mult Scler 2014; Berger and Fox, Journal of neurovirology 2016; 22(4): 536-7; and Journal of Neurovirology 2016; 22(4): 533-5). Thus, treating neurologists are faced with a tremendous dilemma: Should they withhold one of the most effective therapies from their patients, or should they treat them and risk the occurrence of PML? (Stuve and Cutter, JAMA Neurol 2014; 71(8): 945-6).

It is currently impossible to precisely predict patients with MS who are at high risk for PML, and to exclude them from natalizumab therapy. The occurrence of PML during natalizumab treatment in patients with MS is a relevant and a problematic issue for several reasons: (1) Natalizumab is an effective therapy that can greatly diminish the frequency of clinical relapses, and the accumulation of lesions in the brain on neuroimaging (Miller et al., N Engl J Med 2003; 348(1): 15-23; Polman et al., N Engl J Med 2006; 354(9): 899-910; Rudick et al., N Engl J Med 2006; 354(9): 911-23); (2) the substantial risk of PML very likely reduces the number of patients and clinical providers who prescribe this effective agent; (3) there is currently no biological or biochemical marker that allows the identification of PML in at-risk individuals with sufficient precision; and (4) risk-stratification algorithms have not reduced the incidence of PML under natalizumab (Cutter and Stuve, 2014; Berger and Fox, Erratum to: Reassessing the risk of natalizumab-associated PML. J Neurovirol 2016; and Reassessing the risk of natalizumab-associated PML. J Neurovirol 2016).

An alternative strategy to allow patients the benefits of natalizumab therapy without the fear of devastating outcomes from PML is to use CNS immune-reconstitution after its onset as a feasible option. Antagonism of α4-integrin interferes with immune competence of the CNS on multiple levels. Leukocyte migration from the periphery into the CNS involves multiple steps (Ransohoff et al., Nature reviews Immunology 2003; 3(7): 569-81; Luster et al., Nature immunology 2005; 6(12): 1182-9; Holman et al., Biochimica et biophysica acta 2011; 1812(2): 220-30). Integrins, including α4-integrin, facilitate leukocyte migration across the basement membrane of blood vessels and across the extracellular matrix (Kunkel et al., Journal of immunology 2000; 164(6): 3301-8; Bauer et al., Proceedings of the National Academy of Sciences of the United States of America 2009; 106(6): 1920-5). In EAE, the model of MS, an early event is the presentation of antigen in the context of major histocompatibility complex (MHC) II in secondary lymphoid organs to CD4⁺ T helper cells. These CD4⁺ T cells become activated, clonally expand, are can be termed “autoimmune-prone”. They leave the draining lymph nodes, and are now capable of adhering to the endothelium of blood vessel walls and migrating into the CNS. Within the CNS, antigen-specific CD4⁺ T cells are re-activated through the presentation of an identical or similar antigen by perivascular APCs, including hematopoietic macrophages (Hickey and Kimura, Science 1988; 239(4837): 290-2) and DCs (Greter et al., Nature medicine 2005; 11(3): 328-34). At this stage, these CD4⁺ T cells are considered “autoimmune”, and they can initiate and perpetuate CNS inflammation through the secretion of soluble inflammatory mediators, and through attracting other immune-competent leukocytes into the CNS. Antigen-recognition of neurotropic pathogens to T cells in secondary lymphoid organs, their migration to the CNS, and their re-activation in the CNS upon encounter of the pathogen antigen there relies on the same basic immunological principles as CNS autoimmunity.

There is also currently no effective anti-viral agent for JCV, including JCV antigen-specific immunotherapy that would help to eliminate the virus from the CNS. An alternative treatment strategy for PML under natalizumab may be to rapidly re-establish immune surveillance in the CNS in an antigen-independent manner by activating innate immune responses. The rationale for immune-reconstitution therapy in viral diseases is the activation and expansion of T cells. Antiviral innate immunity in the CNS and other organs is mediated by different sensors that detect viral-pathogen-associated molecular patterns (PAMPs) (Hussain et al., Journal of neuroimmunology 2014; 276(1-2): 9-17). Viral double-stranded RNA (dsRNA) is recognized as a PAMP by Toll-like receptor 3 (TLR3) (Tabeta et al., Proceedings of the National Academy of Sciences of the United States of America 2004; 101(10): 3516-21), and it mediates anti-viral defense by diverse host cells. Engagement of TLR3 results in the activation of interferon (IFN) regulatory factors (IRF), and subsequently to high levels of type I IFN production. Type I IFN has anti-viral effects through the activation of genes that inhibit protein synthesis and viral replication (Honda et al., Immunity 2006; 25(3): 349-60). In addition, the IFNβ possesses immunoregulatory properties, and is approved for treatment of relapsing forms if MS (Yong et al., Neurology 1998; 51(3): 682-9).

Immuno-reconstitution therapy in viral diseases aims to activate and expand adaptive immune responses. Often, the initial responses of a host to a viral infection is the synthesis of type I interferons (IFN). Engagement of TLR3 results in the activation of IFN regulatory factors (IRF), and subsequently to high levels of type I IFN production. Type I IFN have both anti-viral and immunoregulatory properties. It was hypothesized that TLR3 agonism in the setting of relative α4-integrin deficiency can re-establish CNS immune surveillance. Disclosed herein are the effects of TLR3 agonism on CNS immune re-constitution in the setting of relative α4-integrin deficiency. It was hypothesized that agonism of TLR3 with polyinosinic-polycytidylic acid (poly I:C) would fully re-establish EAE disease activity in mice that lack α4-integrin.

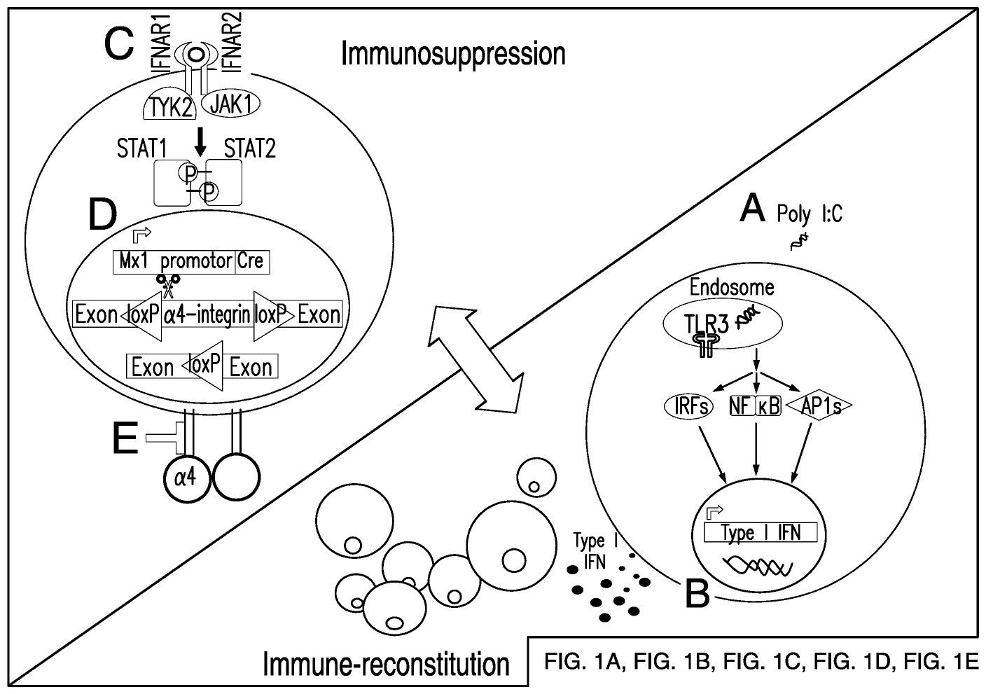

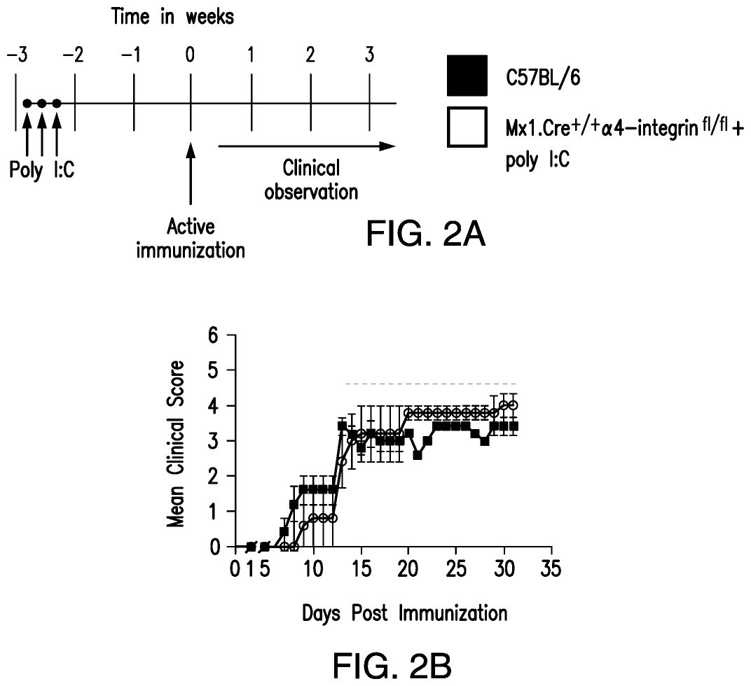

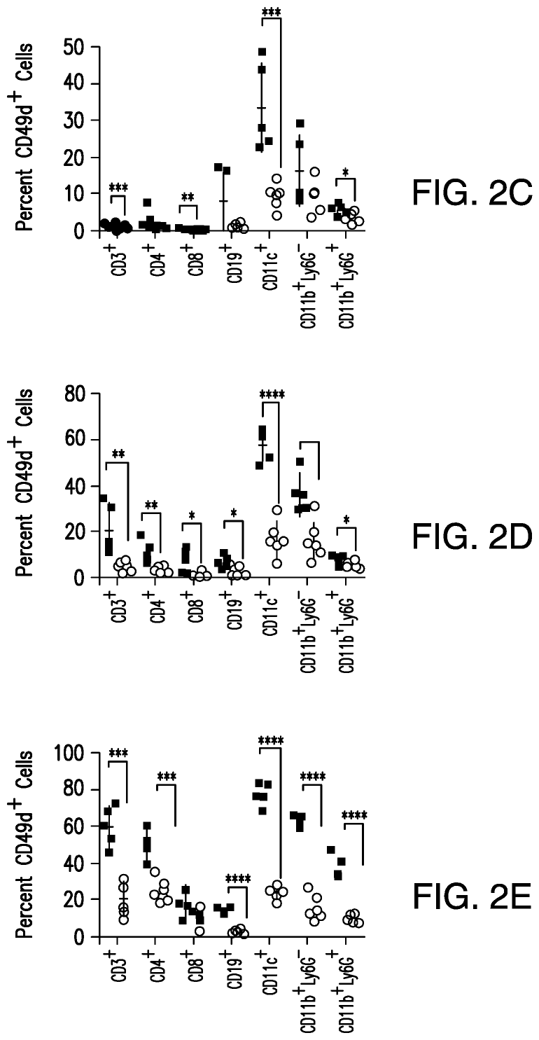

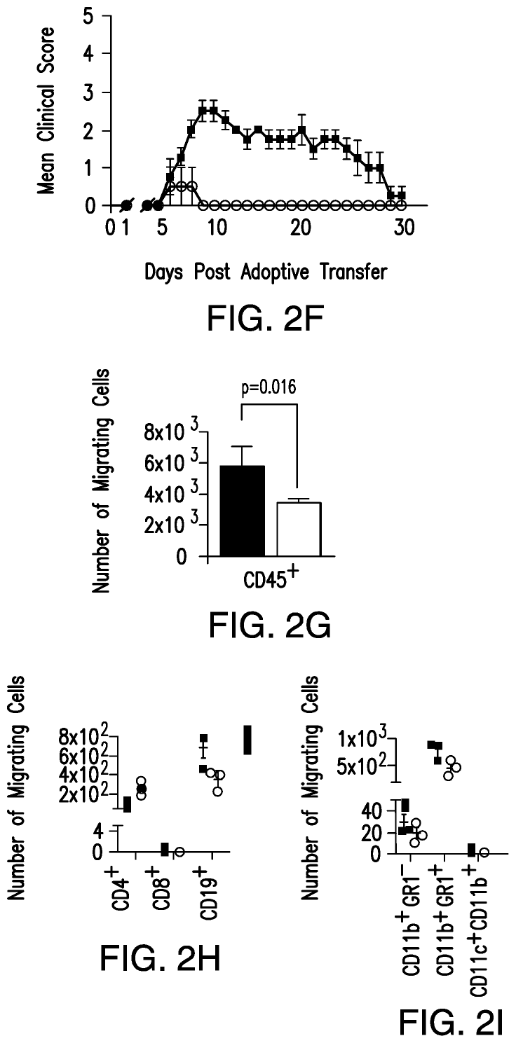

To test this hypothesis, a Mx1.Cre⁺α4-integrin^{flfl} mouse strain was generated. In these mice, the Cre recombinase is under the control of the Mx1 promoter which can be induced to high levels by administration of poly I:C. Poly I:C engagement of TLR3 results in the expression of type I interferons, which subsequently bind IFN type I receptors in adjacent IFN Type I receptor-expressing cells. Consequently, downstream transcription factors translocate to the cell nucleus, and start transcription of antiviral genes, including Mx1. In Mx1.Cre⁺α4-integrin^{fl/fl} mice, Cre recombinase targets loxP sites flanking the Itga4 (α4-integrin) gene, causing its deletion (FIG. 1, FIG. 2A). This system allows the conditional deletion of α4-integrin on IFN-receptor expressing cells, which includes leukocytes. First, the effect of poly I:C on α4-integrin deletion on leukocytes was verified in vivo. Next, the loss of encephalitogenicity of CD4⁺ donor T cells from poly I:C-treated Mx1.Cre⁺α4-integrin^{flfl} mice was confirmed in the adoptive transfer EAE model. To complete the assessment of α4-integrin deletion on leukocyte function and migratory behavior, the effect poly I:C in the setting of α4-integrin deletion on leukocyte migration and proliferation assays was tested in vitro. To test the role of TLR3 agonism on the re-establishment of CNS immune competence, EAE was induced by active immunization of poly I:C treated Mx1.Cre⁺α4-integrin^{flfl} and C57BL/6 wild-type (WT) mice, and by immunophenotyping of leukocytes subsets in secondary lymphoid tissues and the CNS. Integrity of the blood-brain barrier was tested through intravenous injection of Evans Blue dye, and the expression of cytokine was determined by quantitative polymerase chain reaction (qPCR).

The data disclosed herein indicate that TLR3 agonism in the setting of relative α4-integrin deficiency can re-establish CNS immune surveillance, and may present a feasible treatment strategy for PML under natalizumab.

Methods

Disclosed herein are methods of treating multiple sclerosis in a subject. In an aspect, the method comprises (a) identifying a subject in need of treatment; and (b) administering to the subject a therapeutically effective amount of natalizumab and a toll-like receptor 3 (TLR3) agonist. In an aspect, the subject or patient can be a human.

Disclosed herein are methods of inducing interferons in a subject. In an aspect, the method comprises (a) identifying a subject in need of treatment; and (b) administering to the subject a therapeutically effective amount of a toll-like receptor 3 (TLR3) agonist alone or in combination with natalizumab. In an aspect, the subject or patient can be a human.

In an aspect, the TLR3 agonist can be rintatolimod, polyinosinic-polycytidylic acid (Poly (I:C)), or poly-L-lysine (Poly ICLC).

Rintatolimod (also referred to as Ampligen) is an immunomodulatory double-stranded RNA drug that has been shown to protect and stimulate the innate immune system by binding and activating TLR3 receptors.

Polyinosinic-polycytidylic acid is an immunostimulant. Poly (I:C) is structurally similar to double-stranded RNA and can stimulate TLR3. It has a mismatched double-stranded RNA wherein one strand is a polymer of inosinic acid and the other strand is a polymer of cytidylic acid. In some aspects, derivatives of Poly (I:C) can be administered to stimulate TLR3 receptors. For instance, Poly ICLC is a derivative of Poly (I:C). Poly ICLC has been shown to have increased stability in body fluids and reduced toxicity profile.

In an aspect, the multiple sclerosis can be relapsing remitting, secondary progressive, or primary progressive or chronic progressive multiple sclerosis.

In an aspect, the administration of natalizumab can be before, during or after the administration of the Poly (I:C). In some aspects, natalizumab can be administered as a fragment thereof.

Natalizumab (also known as Tysabri®) is a humanized monoclonal antibody against adhesion molecule 4 α4-integrin. Generally, natalizumab can be administered via intravenous infusion about every 28 days or once per month. The mechanism of action of natalizumab is unclear, but it is thought to prevent immune cells (e.g., white blood cells, leukocytes) from crossing blood vessel walls to move into or reach organs.

In an aspect, the subject has been identified as being at risk for progressive multifocal leukoencephalopathy prior to the administering step of the TLR3 agonist. In some aspects, the subject has been diagnosed with progressive multifocal encephalopathy (PML). In some aspects, the subject has been diagnosed with PML prior to the administering step of the TLR3 agonist. Any method known to one of ordinary skill in the art to identify a subject at risk for PML can be used. Examples of methods that can be used to identify a subject at risk for PML include but are not limited to assays for detecting the presence of JC virus antibodies in a biological fluid, for example, serum, plasma or cerebral spinal fluid; determining the percent inhibition in an anti-JC virus antibody confirmation assay. The method can further include determining a JC virus antibody titer. In some aspects, the subject can be identified as being at risk for PML if the antibody titer is above a pre-determined level. In some aspects, the subject can be identified as being at risk for PML if the percent inhibition is below a pre-determined level. In some aspects, the subject has been identified as being at risk for PML because the patient has or is currently receiving an immunosuppressant therapy. In some aspects, the subject has been identified as being at risk for PML because the patient has or is currently receiving a natalizumab or a fragment thereof.

Progressive multifocal leukoencephalopathy is an opportunistic infection caused by the JC virus. PML can be a fatal disease. PML can be characterized by progressive damage or inflammation of the white mater of the brain at two or more locations. Symptoms of PML can develop over time, for example over a period of one or more weeks to one or more months. The symptoms of PML can depend on the location of the damage in the brain and the degree or extend of the damage. Symptoms can include one or more of the following: clumsiness, progressive weakness, visual, speech and personality changes. Generally, the JC virus can be harmless unless it is present in a subject with a weakened immune system. JC can be present under normal conditions and kept under control of the immune system. PML can be diagnosed in subjects with a severe immune deficiency, for example, subjects with acquired immune deficiency syndrome. PML can also be diagnosed in subjects that are administered immunosuppressive medications, including chemotherapeutic agents. Other subjects at risk include but are not limited to subjects with transplants, Hodgkin's lymphoma, multiple sclerosis, psoriasis and other immune diseases.

PML can be diagnosed in a subject with a progressive course of the disease. Examples of diagnosing PML include but are not limited to: confirmation of JC virus DNA in spinal fluid along with 1) white matter brain lesions from magnetic resonance imaging; or 2) a brain biopsy showing demyelination, abnormal astrocytes and/or the presence of enlarged oligodendroglial nuclei. While any area of the brain can be affected or damaged, common areas of lesions include but are not limited include frontal and parietooccipital lobes. Additionally, gray matter brain areas can also have lesions and include the basal ganglia. Further, lesions can be present in the external capsule, posterior cranial fossa, brainstem and cerebellum.

In an aspect, the method can further comprise monitoring the subject for indicators of progressive multifocal leukoencephalopathy. Methods of monitoring subjects for indicators of PML can be associated with the progression of clinical disease. For instance, in subjects with no clinical disease progression, for example, multiple sclerosis, the interval of monitoring the subject can be annually. In subjects that that a history and/or a physical exam that indicates progression of a clinical disease such as MS or symptoms and/or signs of a potential opportunistic disease process, a screening test can be carried out to identify whether the clinical disease progression is due to a central nervous system opportunistic disease process. The monitoring can include performing MRI brain images, evaluating cerebrospinal fluid. The interval for monitoring the subject for indicators of PML can be every two weeks to every two to three weeks following a positive test indicating PML. Interval monitoring can continue for as long as the subject is treated with natalizumab or a fragment thereof. Symptoms of PML include but are not limited to weakness or paralysis, vision loss, impaired speech, and cognitive deterioration

In an aspect, natalizumab can be administered intravenously. In some aspects, the natalizumab can be administered to the subject over a series of treatments.

In an aspect, the Poly (I:C), Poly ICLC or rintatolimod can be administered intravenously or intranasally. In some aspects, the Poly (I:C), Poly ICLC or rintatolimod can be administered to the subject over a series of treatments.

Disclosed herein are methods of treating a patient at risk of having progressive multifocal leukoencephalopathy (PML). The method can comprise administering to a patient a therapeutically effective amount of a toll-like receptor 3 (TLR3) agonist. In an aspect, the patient has undergone therapy with natalizumab. In some aspects, the patient has been diagnosed with multiple sclerosis. In an aspect, the multiple sclerosis can be relapsing remitting, secondary progressive, or primary progressive or chronic progressive multiple sclerosis. In some aspects, the patient has been diagnosed with Crohn's disease. In an aspect, the TLR3 agonist can be rintatolimod, polyinosinic-polycytidylic acid (Poly (I:C)), or poly-L-lysine (Poly ICLC).

Disclosed herein are methods of treating a patient at risk of having progressive multifocal leukoencephalopathy (PML). The method can comprise administering to a patient a population of activated T cells. In an aspect, the population of T cells can be activated in vitro with an immunotherapeutic agent that can stimulate a toll-like receptor 3 (TLR3). In an aspect, the immunotherapeutic agent can be Poly (I:C). In an aspect, the immunotherapeutic agent can be rintatolimod or poly-L-lysine (Poly ICLC). In some aspects, procedures and/or methods of administering TLR3 agonists are taught in Neurology, 1986; 36:494-498. In some aspects, the patient has been diagnosed with multiple sclerosis. In an aspect, the multiple sclerosis can be relapsing remitting, secondary progressive, or primary progressive or chronic progressive multiple sclerosis. In some aspects, the patient has been diagnosed with Crohn's disease. In some aspects, the patient has undergone therapy with natalizumab. In an aspect, the method can further comprise monitoring the patient for indicators of PML. In an aspect, the population of T cells can be administered intravenously.

Disclosed herein are methods of activating T cells in a subject. The method can comprise administering a therapeutically effective amount of a composition comprising polyinosinic-polycytidylic acid (Poly (I:C)) to a subject having or suspected of having a reduced number of T cells. In some aspects, the method can comprise administering a therapeutically effective amount of a composition comprising rintatolimod, polyinosinic-polycytidylic acid (Poly (I:C)), or poly-L-lysine (Poly ICLC). In an aspect, the subject has previously undergone treatment with natalizumab. In an aspect, the subject can be currently undergoing treatment with natalizumab. In an aspect, the number of T cells can be increased after administration of the Poly (I:C), rintatolimod or Poly ICLC. In some aspects, the patient has been diagnosed with multiple sclerosis. In an aspect, the multiple sclerosis can be relapsing remitting, secondary progressive, or primary progressive or chronic progressive multiple sclerosis. In some aspects, the patient has been diagnosed with Crohn's disease. In some aspects, the patient has undergone therapy with natalizumab. In an aspect, the method can further comprise monitoring the patient for indicators of PML. In an aspect, the population of T cells can be administered intravenously.

In some aspects, the method can include administering to a subject a population of T cells activated and expanded, wherein the population of T cells was activated in vitro with an agent that can stimulate TLR3 receptors. The activating and stimulating steps can thereby induce proliferation of the T cells. In an aspect, the agent can be a TLR3 receptor agonist.

As used herein, the term, “activating T cells” refers to a ligand which recognizes and binds with a cognate binding partner (e.g., a stimulatory and/or costimulatory molecule present on a T cell) protein present in a sample, but which the ligand does not substantially recognize or bind other molecules in the sample. “Activation”, as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

By the term “stimulation,” is meant a primary response induced by binding of a stimulatory molecule with its cognate ligand thereby mediating a signal transduction event. Stimulation can mediate altered expression of certain molecules.

Prior to expansion, a source of T cells can be obtained from a subject. T cells can be obtained from a number of sources, including but not limited to peripheral blood mononuclear cells, bone marrow, lymph node tissue, cord blood, thymus tissue, tissue from a site of infection, ascites, pleural effusion, spleen tissue, and tumors. In certain aspects, any number of T cell lines available in the art, may be used. In some aspects, T cells can be obtained from a unit of blood collected from a subject using any number of techniques known to one of ordinary skill in the art, such as ficoll separation. In an aspect, cells from the circulating blood of a subject can be obtained by apheresis or leukapheresis. The apheresis product typically contains lymphocytes, including T cells, monocytes, granulocytes, B cells, other nucleated white blood cells, red blood cells, and platelets. In an aspect, the cells can be collected by apheresis and may be washed to remove the plasma fraction and to place the cells in an appropriate buffer or media for subsequent processing steps. In an aspect, the cells can be washed with phosphate buffered saline (PBS). In an aspect, the wash solution can lack calcium and magnesium or may lack many if not all divalent cations. Initial activation steps in the absence of calcium can lead to magnified activation. A washing step can be accomplished by methods known to those in the art, such as by using a semi-automated “flow-through” centrifuge. After washing, the cells can be resuspended in a variety of biocompatible buffers, such as, for example, Ca-free, Mg-free PBS, PlasmaLyte A, or other saline solution with or without buffer. Alternatively, the undesirable components of the apheresis sample can be removed and the cells directly resuspended in culture media.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like.

A “stimulatory molecule,” as the term is used herein, means a molecule on a T cell that specifically binds with a cognate stimulatory ligand present on an antigen presenting cell.

Disclosed herein are methods of preventing progressive multifocal leukoenchephalopathy (PML) in a subject. In an aspect, the subject has multiple sclerosis or Crohn's disease. The method can comprise: (a) identifying a subject in need of treatment; and

(b) administering to the subject a therapeutically effective amount of a toll-like receptor 3 agonist before, during or after administration of natalizumab, in an amount sufficient to prevent PML.

Compositions

The compositions described herein can be formulated to include a therapeutically effective amount of natalizumab and TLR3 agonist as described herein. Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a type of disease, disorder or infection.

The compositions described herein can be formulation in a variety of combinations. The particular combination of natalizumab and a toll-like receptor 3 (TLR3) agonist can vary according to many factors, for example, the particular the type and severity of MS, Crohn's disease, and/or risk for PML or any combination thereof.

The compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions are administered to a subject (e.g., a human patient) already with or diagnosed with multiple sclerosis, Crohn's disease or PML in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the multiple sclerosis, Crohn's disease or PML is delayed, hindered, or prevented, or the multiple sclerosis, Crohn's disease or PML or a symptom of the multiple sclerosis, Crohn's disease or PML is ameliorated. One or more of the symptoms can be less severe. Recovery can be accelerated in an individual who has been treated.

Disclosed herein, are methods of treating a patient with multiple sclerosis. The multiple sclerosis can be any category or classification of multiple sclerosis. In some aspects, the multiple sclerosis can be relapsing remitting, secondary progressive, or primary progressive multiple sclerosis. In an aspect, the subject has been diagnosed with multiple sclerosis prior to the administering step.

Disclosed herein, are methods of treating a patient at risk of having progressive multifocal leukoencephalopathy. In an aspect, the patient has been diagnosed with multiple sclerosis prior to the administering step.

The compositions described herein can be formulated to include a therapeutically effective amount of natalizumab alone or in combination with one of the toll-like receptor 3 agonists disclosed herein.

The therapeutically effective amount or dosage of natalizumab, and toll-like receptor 3 agonists used in the methods as disclosed herein applied to mammals (e.g., humans) can be determined by one of ordinary skill in the art with consideration of individual differences in age, weight, sex, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the clinical disease symptoms), the age and physical characteristics of the subject and other considerations known to those of ordinary skill in the art. Dosages can be established using clinical approaches known to one of ordinary skill in the art.

The duration of treatment with any composition provided herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

Dosages of natalizumab can be in about 300 mg intravenously (i.v.) every 28 days regular dosing to about about 300 mg iv every 56 days extended dosing, or any amount in between.

Dosages of rintatolimod can be in the range of about 100 mg per i.v. weekly to about 400 mg i.v. twice weekly or any amount in between.

Dosages of polyinosinic-polycytidylic acid can be in the range of about 20 μg/kg body weight i.v. weekly to about 100 μg/kg body weight i.v. weekly or any amount in between.

The amount specified can be the amount administered as the average daily, average weekly, or average monthly dose, or it may be expressed in terms of mg/kg, where kg refers to the weight of the patient and the mg is specified above. A clinician can readily determine the effective amount of any of the compounds disclosed herein by taking into account factors, such as the size and weight of the subject; the extent of disease penetration; the age, health and sex of the subject; the route of administration; and whether the administration is regional or systemic.

The total effective amount of the compositions as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

The compositions described herein can be administered in conjunction with other therapeutic modalities to a subject in need of therapy. Natalizumab can be given prior to, simultaneously with or after treatment with a toll-like receptor 3 agonist. In an aspect, natalizumab can be given prior to, simultaneously or during, or after administration of a toll-like receptor agonist.

In an aspect, natalizumab can be co-formulated with any of the toll-like receptor agonists disclosed herein.

Natalizumab can be administered as “combination” therapy. It is to be understood that, for example, natalizumab can be provided to the subject in need, either prior to administration of a toll-like receptor 3 agonist, concomitant with administration of said toll-like receptor 3 agonist (co-administration) or shortly thereafter.

As disclosed herein, are compositions that can be formulated for parental administration. In an aspect, the parental administration can be intravenous, subcutaneous, intramuscular or direct injection. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The compositions can be administered directly to a subject. Generally, the compositions can be suspended in a pharmaceutically acceptable carrier (e.g., physiological saline or a buffered saline solution) to facilitate their delivery.

The compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, and the like can also be used.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used. Thus, compositions can be prepared for parenteral administration that includes for example, natalizumab or rintatolimod dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules.

Articles of Manufacture

The composition described herein can be packaged in a suitable container labeled, for example, for use as a therapy to treat multiple sclerosis, Crohn's disease or any of the methods disclosed herein. Accordingly, packaged products (e.g., sterile containers containing the composition described herein and packaged for storage, shipment, or sale at concentrated or ready-to-use concentrations) and kits, including at least a TLR3 agonist as described herein and instructions for use, are also within the scope of the disclosure. A product can include a container (e.g., a vial, jar, bottle, bag, or the like) containing the composition described herein. In addition, an article of manufacture further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio- or videotape)). The legend can be associated with the container (e.g., affixed to the container) and can describe the manner in which the compound therein should be administered (e.g., the frequency and route of administration), indications therefor, and other uses. The compounds can be ready for administration (e.g., present in dose-appropriate units), and may include a pharmaceutically acceptable adjuvant, carrier or other diluent. Alternatively, the compounds can be provided in a concentrated form with a diluent and instructions for dilution.

### EXAMPLES

### Example 1: In the Absence of Poly I:C, Mx1.Cre⁺α4-Integrin^{fl/fl} Mice Behave Like Control Animals

As stated herein, experiments were carried out to test whether TLR3 agonism leads the CNS immune-reconstitution in the setting of relative α4-integrin deficiency. The results described herein show that EAE disease susceptibility can be fully re-established when active EAE is induced in the setting of poly I:C-mediated conditional deletion of α4-integrin in the Mx1.Cre⁺α4-integrin^{flfl} mice, which is a functional read-out that confirms intact adaptive immune responses within the CNS. These data indicate that immune-competence could also be re-established in the setting of a CNS infection, for instance in patients with PML under natalizumab.

Given that recombinant type I IFNs are already approved for human disease, including for MS, it is thought that TLR3 agonism would prove to be beneficial because exogenous type I IFN does not have good CNS bioavailability in the brain (Cathala and Baron, J Immunol 1970; 104(6): 1355-8; Habif et al., Proc Soc Exp Biol Med 1975; 149(1): 287-9; Aguet, Nature 1980; 284(5755): 459-61; Vass and Lassmann, The American journal of pathology 1990; 137(4): 789-800). In fact, findings by Field et al demonstrate that systemic administration of poly I:C leads to an upregulation of type I IFNs in the CNS with of mice (Field et al., Brain, behavior, and immunity 2010; 24(6): 996-1007).

A potential concern in pursuing TLR3-mediated immune re-constitution in the setting of PML under natalizumab is inflammatory immune reconstitution syndrome (IRIS) of the brain, which in itself can result in devastating neurological outcomes (Dahlhaus et al., Journal of neurology, neurosurgery, and psychiatry 2013; 84(10): 1068-74; Fine et al., Annals of neurology 2014; 75(1): 108-15). The induction of endogenous type I IFN expression through TLR3 agonism would possibly provide a strong anti-viral effect as well as some degree of immunomodulation. As stated above, several IFNβ-1a and IFNβ-1b preparations are currently approved for the treatment of MS because of their anti-inflammatory properties (Yong et al., 1998).

Finally, the concept of TLR3 agonism in the setting of PML or patients at risk for PML can be tested in a clinical setting. For instance, the TLR3 agonist rintatolimod has already been tested in several phase III trials for the treatment of chronic fatigue syndrome/myalgic encephalomyelitis (CFS/ME), and were found to be safe.

Conclusively, the data described herein provides a biological rationale to test TLR3 agonists in patients with MS who develop PML under natalizumab.

The hypothesis that agonism of TLR3 with polyinosinic-polycytidylic acid (poly I:C) would fully re-establish EAE disease activity in mice that lack α4-integrin was tested. To address this hypothesis, the Mx1.Cre⁺α4-integrin^{flfl} mouse strain was generated as described herein. In these mice, the Cre recombinase is under the control of the Mx1 promoter which can be induced to high levels by administration of poly I:C. In Mx1.Cre⁺α4-integrin^{fl/fl} mice, Cre recombinase targets loxP sites flanking the Itga4 (α4-integrin) gene, causing its deletion. See, FIG. 1A-E and FIG. 2A.

To ascertain that Mx1.Cre⁺α4-integrin^{flfl} mice do not possess any biological properties that would impact the subsequent experiments, active EAE was induced in the absence of poly I:C (FIG. 2B). EAE disease incidence, onset, and clinical severity were similar between Mx1.Cre⁺α4-integrin^{fl/fl} mice and C57BL/6 control mice.

Mice.

C57BL/6J mice and Mx1.Cre⁺ (B6.Cg-Tg(Mx1-cre)1Cgn/J) were purchased from The Jackson Laboratory, Bar Harbor, Me., USA (Kuhn et al., Science 1995; 269(5229): 1427-9). α4^{fl/fl} mice were generated, described, and obtained from Dr. Thalia Papayannopoulou, University of Washington (Scott et al., Molecular and cellular biology 2003; 23(24): 9349-60). Briefly, a targeting vector was constructed including the promoter and first two exons of α4 integrin gene, a PGK-neo-p(A) cassette flanked by loxP elements, with an additional loxP inserted distal to the second exon. AK7 cells were electroporated with linearized vector and floxed clones resulted from homologous recombination. α4^{floxed} clones were identified with specific primers. Clones with normal XY karyotype were injected into C57BL/6 blastocysts and transferred into pseudo pregnant females. Resulting male chimeras were then bred to C57BL/6 females. Offspring were genotyped and animals heterozygous for the floxed α4 allele were crossed to generate floxed homozygotes.

α4^{fl/fl} females were bred to Mx1.Cre⁺ males. Progeny were genotyped for the cre transgene by PCR utilizing generic cre primers (5′-GTGAAACAGCATTGCTGTCACTT-3′ (SEQ ID NO: 1) and 5′-GCGGTCTGCCAGTAAAAATATC-3′ (SEQ ID NO: 2)). Mx1.Cre⁺α4^{fl/+} mice were intercrossed, and Mx1.Cre⁺ progeny were genotyped for the α4 allele (5′-GTCCACTGTTGGGCAAGTCC-3′ (SEQ ID NO: 3) and 5′-AAACTTGTCTCCTCTGCCGTC3′ (SEQ ID NO: 4)). Eight to twelve weeks old, both female and male mice were used for all experiments. Mx1.Cre⁺α4^{fl/fl} mice received 3 intra peritoneal injections of 300 μg poly(I)-poly(C) (poly I:C; Sigma Chemical Company, St. Louis, Mo.) given at 2 days intervals in order to activate the Cre recombinase. This was followed by a “wash-out” period of three weeks in which mice were then analyzed or immunized for EAE.

All mice described in this work were crossed and maintained in a pathogen-free animal facility.

Active Induction of EAE. Mice were anesthetized with 200 mg/kg tribromomethanol (1.5% Avertin) injected i.p. Active EAE was induced by s.c. injections into the flanks with 200 μg of mouse myelin oligodendrocyte glycoprotein₃₅₋₅₅ (MOGₚ₃₅₋₅₅) (MEVGWYRSPFSRVVHLYRNGK (SEQ ID NO: 5); CS Bio Menlo Park, Calif., USA) emulsified in complete Freund's adjuvant (CFA) (DIFCO Laboratories, Detroit, Mich., USA) containing 400 μg of heat inactivated Mycobacterium tuberculosis (Difco, Detroit, Mich., USA). Mice also received i.p. injections of 200 ng pertussis toxin on days 0 and 2 (List Biological Laboratories Inc., Campbell, Calif., USA). Clinical signs of EAE were assessed daily and reported following the classical criteria: 0=no clinical disease, 1=limp tail, 2=partial hind leg paralysis, 3=complete hind leg and uni-lateral paralysis, 4=complete hind leg and partial front leg paralysis, 5=moribund (Cravens et al., Journal of neuroinflammation 2013; 10: 67). At least three independent experiments were conducted with a minimum of five mice per group.

Statistical Analysis.

All experiments were repeated at least twice and at least 5 mice were utilized per treatment group. For parametric tests, data were checked for normality by using the Kolmogorov-Smirnov test. The means of samples were compared using an unpaired Student's t-test. Mean clinical scores significance between groups was analyzed by Mann-Whitney Utest. The criterion for significance (alpha) has been set at *p<0.05, **p<0.01, ***p<0.001, ****p<0.0001. Data are given as mean±standard error. All analyses were performed with Prism 6 for Windows (GraphPad Software, La Jolla, Calif., USA).

### Example 2: The Frequency α4-Integrin (CD49d)-Positive Leukocytes is Reduced in Primary and Secondary Lymphoid Organs of Poly I:C-Treated Mx1.Cre⁺α4-Integrin^{fl/fl} Mice

The frequency of α4-integrin (CD49d)-positive leukocyte subsets was assessed by multi-parameter flow cytometry in poly I:C-treated Mx1.Cre⁺α4-integrin^{fl/fl} mice, and in poly I:C-treated C57BL/6 control mice on day 15 post active EAE induction. FIGS. 2A-2I show that these cells have a decreased ability to adoptively transfer EAE and to migrate across biological membranes. Then, the frequency of α4-integrin (CD49d)-expressing leukocyte subsets was assessed by multi-parameter flow cytometry in poly I:C-treated Mx1.Cre⁺α4-integrin^{fl/fl} mice, and in poly I:C-treated C57BL/6 control mice on day 15 post active EAE induction. In the lymph nodes (FIG. 2C), spleen (FIG. 2D), and bone marrow (FIG. 2E) of poly I:C-treated Mx1.Cre⁺α4-integrin^{fl/fl} mice, the frequency of α4-integrin (CD49d)-expressing CD3⁺ T cells, CD8⁺ T cells, CD11c⁺ monocyte-derived dendritic cells (DC), and CD22b⁺ Ly6G⁺ myeloid-derived granulocytes was significantly diminished. In spleen (FIG. 2D), and bone marrow (FIG. 2E), the frequency of α4-integrin expressing CD4⁺ T cells, CD19⁺ B cells, and CD22b⁺ Ly6G⁻ macrophages was also significantly reduced. Similar trends for the latter cell subsets were seen in lymph nodes but do not reach statistical significance (FIG. 2C).

Immunophenotyping.

1×10⁶ cells from spleens, lymph nodes, bone marrow, and CNS were resuspended in FACS buffer (5% Fetal Bovine Serum (FBS) in PBS) and Fc receptors were blocked with anti-CD16/32 (Tonbo Biosciences) for 10 minutes at 4° C. For blood analysis, 60 μl of blood were also treated with 1 μg anti-CD16/32 but incubated and stained at room temperature. Cells were then stained for surface markers with fluorochrome-conjugated mAbs: Integrin α4-FITC from Santa Cruz Biotechnology, Inc.; CD3e-Pacific Blue, CD19-Alexa Fluor 700, CD11c-PE, CD11b-APC, GR-1-APC-Cy7 all from BD Biosciences (San Jose, Calif., USA); CD45-PE-Cy7 from eBioscience (San Diego, Calif., USA); CD4-PE-Texas Red, CD8-Pacific Orange both from Invitrogen (Grand Island, N.Y., USA); CD11a-PE and CD49e-PE from BioLegend (San Diego, Calif., USA); biotinylated PDCA-1 from Miltenyi (Auburn, Calif., USA) which was revealed with SA-Q Dot 655 from Invitrogen (Grand Island, N.Y., USA). Cells were then washed, resuspended in staining buffer, and fixed in 0.5% paraformaldehyde.

Fluorescence minus one (FMO) controls were prepared by adding all antibodies but one, for each parameter to discriminate positive staining from non-specific background. Up to 500,000 events were acquired on a BD FACS LSR II at UT Southwestern Flow Core or FACS LSRFortessa SORP at The Moody Foundation Flow Cytometry Facility, Children's Medical Center Research Institute at UT Southwestern. Data was analyzed using FlowJo software (Tree Star, Ashland, Oreg., USA).

### Example 3: Adoptively Transfer of Lymph Node Cells from Poly I:C-Treated Mx1.Cre⁺α4-Integrin^{fl/fl} Donor Mice Results in Ameliorated EAE Disease Activity

Next, the in vivo role of genetic α4-integrin ablation was tested by passively transferring activated cells from myelin oligodendrocyte glycoprotein peptide (MOGₚ) 35-55-immunized poly I:C-treated Mx1.Cre⁺α4-integrin^{fl/fl} mice, or poly I:C-treated C57BL/6 mice into naïve C57BL/6 recipient mice. In the adoptive transfer model, the recipient mice are not exposed to the effects of poly I:C. Transfer of cells from both strains resulted in the onset of clinic disease at day 7 (FIG. 2F). However, transfer of poly I:C-treated Mx1.Cre⁺α4-integrin^{fl/fl} donor T cells was associated with a disease incidence of only 75%, and in a significantly ameliorated and shortened disease course in recipient mice. In this experimental paradigm, recipient mice fully recovered by day 15 post transfer. In contrast, adoptive transfer of poly I:C treated C57BL/6 donor T cells resulted in 100% disease incidence, and a significantly more severe course (FIG. 2F).

Adoptive Transfer EAE.

For passive induction of EAE by adoptive transfer of myelin-specific T cell, single cell suspensions were prepared from splenocytes isolated from actively immunized mice. Cells were activated for 72 hours with MOG₃₅₋₅₅ and IL-12 in vitro (Miller et al., Curr Protoc Immunol 2007; Chapter: Unit-15.1). After incubation, 5 million cells were injected i.p. into C57BL/6 recipients. Clinical signs of EAE were assessed daily and reported following the classical criteria: 0=no clinical disease, 1=limp tail, 2=partial hind leg paralysis, 3=complete hind leg and uni-lateral paralysis, 4=complete hind leg and partial front leg paralysis, 5=moribund.

### Example 4: Splenocytes from Systemically Poly I:C-Treated Mx1.Cre⁺α4-Integrin^{fl/fl} Show Reduced Migratory Capabilities In Vitro

To test the effect of α4-integrin deletion after poly I:C treatment on migratory competence of cells, an in vitro migration assay by Boyden Chamber as described before (Stuve et al., Annals of neurology 1996; 40(6): 853-63) was performed. A significant reduction in the migration of CD45⁺ splenocytes from poly I:C-treated Mx1.Cre⁺α4-integrin^{fl/fl} mice was observed when compared to CD45⁺ splenocytes from poly I:C-treated C57BL/6 mice (FIG. 2G). Further characterization of splenocyte subsets from both mouse strains revealed that there was no significant difference in the migratory capacity of lymphocyte subsets (FIG. 2H), or myeloid cell subsets (FIG. 2I). However, there was a trend towards decreased migratory capacity of CD19⁺ B cells (FIG. 2H), CD11b⁺GR1⁺ granulocytes and CD11c⁺CD11b⁺ DC (FIG. 2I).

Isolation of Lymph Node Cells and Splenocytes.

Lymph node cells and splenocytes were isolated by pressing through a 70 μM nylon mesh cell strainer. Cells were treated with RBC lysis buffer (Sigma-Aldrich, St. Louis, Mo., USA), washed twice with cold PBS, and resuspended in EAE media or PBS for counting with hemocytometer.

In Vitro Migration Assay.

An in vitro migration assay was performed by Boyden Chamber as described before (Stuve et al., Annals of neurology 1996; 40(6): 853-63). Briefly, a Boyden chamber containing a polycarbonate membrane filter (Transwell® Permeable Supports, Corning Inc., Corning N.Y.) pre-coated on its upper surface with 20 μg/ml FN was used. A total of 6×10⁵ splenocytes, suspended in EAE media, were added to the upper chamber. Chambers were then incubated at 37° C. for 6 to 8 hours. Following incubation, the content of the lower chamber was collected, and the number of cells was counted with a hemocytometer and the phenotype of the cells determined by flow cytometry.

### Example 5: In Vivo TLR3 Agonism Through Systemic Poly I:C Treatment Re-Establishes EAE Disease Susceptibility and CNS Immune Competence in the Setting of Relative α4-Integrin Deficiency

After establishing that the frequency α4-integrin (CD49d)-positive leukocytes is significantly reduced in primary and secondary lymphoid organs of poly I:C-treated Mx1.Cre⁺α4-integrin^{fl/fl} mice, and that the capacity of lymphocytes from these mice to induce passively transferred EAE, and to migrate across biological membranes in vitro is substantially diminished, the following experiments demonstrated that in vivo TLR3 agonism through poly I:C treatment reverses the effects of relative α4-integrin deficiency on EAE disease activity. Full EAE susceptibility requires the entry of leukocytes into the brain and spinal cord, and consequently cannot occur in the setting of compromised CNS immune competence.

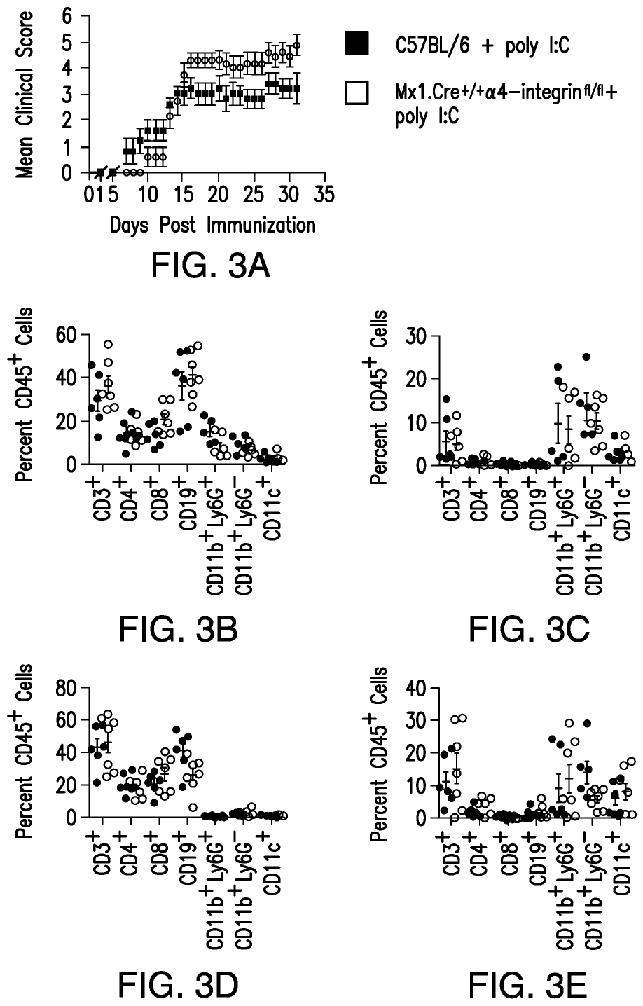

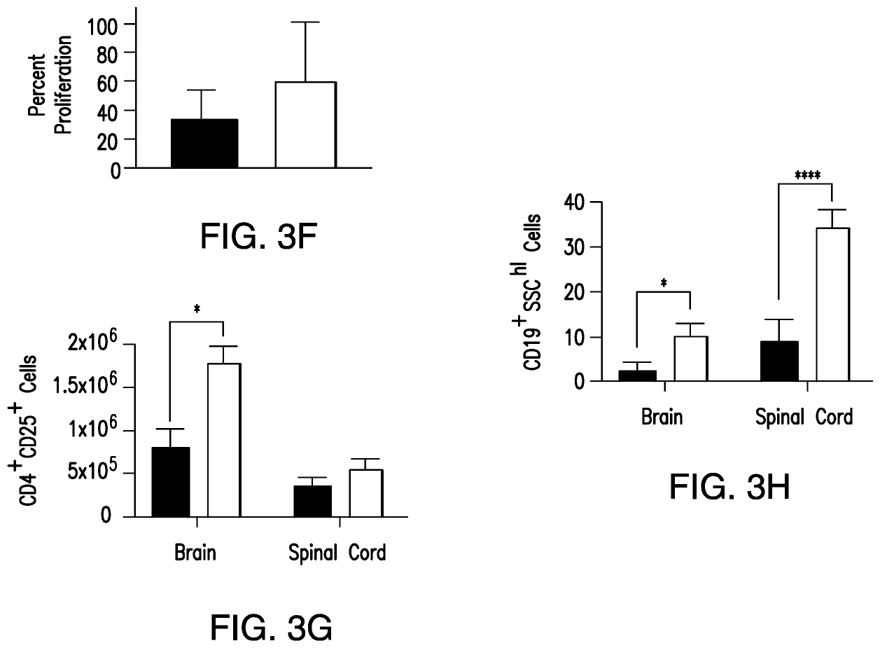

When active EAE was induced in Mx1.Cre⁺α4-integrin^{fl/fl} mice and C57BL/6 control mice that were treated with poly I:C, EAE disease incidence, susceptibility and severity were similar in both groups (FIG. 3A). There was a trend towards more severe clinical EAE disease in Mx1.Cre⁺α4-integrin^{fl/fl} mice treated with poly I:C (FIG. 3A). These results indicate that CNS immune surveillance was functionally re-established in our model.

Enzymatic CNS Digestions.

As previously described (Hussain R Z, Neurology: Neuroimmunology & Neuroinflammation 2017), brains and spinal cords were first finely minced using a sterile scalpel, washed with cold PBS, then processed based on the specific enzymes used. The commercially available Neural Tissue Dissociation Kit (P) (Kit) was used following the manufacturer's protocol (Neural Tissue Dissociation Kit (P), Miltenyi Biotec, San Diego, Calif., USA). Following enzymatic dissociation, brains and spinal cords were washed with cold PBS, and then subjected to one wash with 37% Percoll PLUS™ to remove remaining myelin. The myelin-free single cell suspensions were counted using a hemocytometer.

### Example 6: In Vivo TLR3 Agonism Through Systemic Poly I:C Treatment Leads to Cellular Immune Reconstitution in the Setting of Relative α4-Integrin Deficiency

Next, the percentage of leukocytes in (B) lymph nodes, (C) spleen, (D) brain, and (E) spinal cord was assessed in mice that were actively immunized for EAE and treated with poly I:C on day 15 as shown in FIGS. 3B-E. In all compartments, there were no differences in composition of leukocytes between the two strains, indicating a full cellular immune re-constitution.

### Example 7: In Vivo TLR3 Agonism Through Systemic Poly I:C Treatment is Associated with Activated and Functional Antigen-Specific Lymphocytes in the Setting of Relative α4-Integrin Deficiency

To investigate possible causes of immune reconstitution Mx1.Cre⁺α4-integrin^{fl/fl} mice treated with poly I:C, antigen recall and activation status of CD4⁺ T cells from lymph nodes obtained at day 10 were investigated after active induction of EAE from Mx1.Cre⁺α4-integrin^{fl/fl} mice and C57BL/6 control mice treated with poly I:C. There was no difference between the capacity of Mx1.Cre⁺α4-integrin^{fl/fl} mice and C57BL/6 control mice that were treated with poly I:C to mount recall responses to MOGₚ₃₅₋₅₅ (FIG. 3F). There was a trend towards strong MOGₚ₃₅₋₅₅ CD4⁺ T cell proliferation from Mx1.Cre⁺α4-integrin^{fl/fl} mice treated with poly I:C (FIG. 3F). The number of activated CD4⁺CD25⁺ T cells was increased in the brain of Mx1.Cre⁺α4-integrin^{fl/fl} mice treated with poly I:C, and similar between both mouse strains in the spinal cord (FIG. 3G). In the brain and spinal cord, it was also observed that a significant expansion of CD19⁺SSC^{hi} B cells in Mx1.Cre⁺α4-integrin^{fl/fl} mice treated with poly I:C (FIG. 3H). These cells were not further characterized, but may be plasmablasts. Poly I:C structural analogues are known to promote robust mucosal and systemic IgG antibody synthesis (Bardel E, Npj Vaccines 2016; 1(16010): 1-10).

Proliferation Assay.

Fifteen days post immunization, single cell suspensions were generated by isolating the LNs of the immunized mice. Utilizing the CellTrace™ CFSE (5(6)-carboxyfluorescein N-hydroxysuccinimidyl ester) Cell Proliferation kit (Life Technologies, Carlsbad, Calif.), CD4⁺ T cell proliferation against antigens was determined. Briefly, isolated 20×10⁶ LN cells were incubated for 5 minutes at room temperature with 1 μM CFSE. After incubation, cells were washed with RPMI media twice, then incubated in a 96-well-round bottom plate at 1×10⁶ cells per well with specified antigen for 96 hours. Post incubation, cells were washed with staining FACS buffer two times, then the Fc receptors were blocked with anti-CD16/32 (BD Biosciences, Franklin Lakes, N.J.,) for 15 minutes at 4° C. before staining with mAbs for 30 minutes at 4° C. Cells were stained utilizing the following monoclonal antibodies: CD3e-Pacific Blue, CD45-PE-Cy7 and CD4-PE-Texas Red. Cells were analyzed with a LSRII flow cytometer (BD Biosciences) and FlowJo software (Tree Star, Ashland, Oreg., USA).

### Example 8: TLR3 Agonism Through Poly I:C Treatment Compromises the Blood-Brain Barrier (BBB) in Mx1.Cre⁺α4-Integrin^{fl/fl} Mice

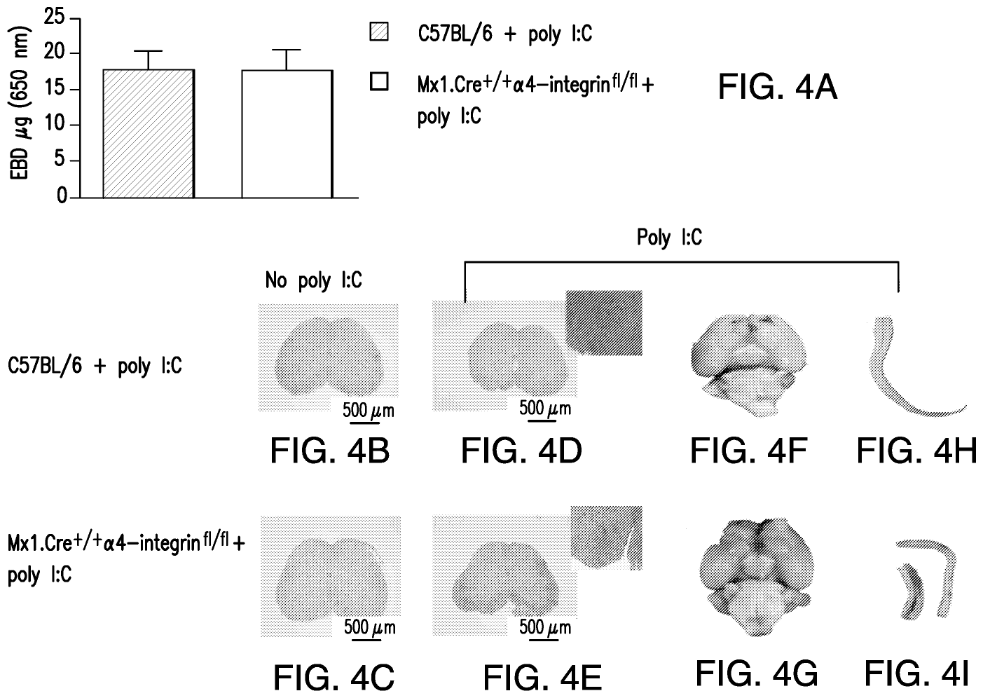

The clinical data and cellular data in Mx1.Cre⁺α4-integrin^{fl/fl} mice and C57BL/6 control mice that were treated with poly I:C indicated that leukocytes are capable of obtaining access to the CNS in the relative absence of α4-integrin when TLR3 is agonized with poly I:C: To test the effect of in vivo poly I:C treatment on blood-brain barrier BBB integrity, an Evans Blue Dye (EBD) permeability assay was performed. EBD has a high affinity for serum albumin. In the setting of BBB compromise, the serum-dye complex can penetrate the CNS parenchyma, and it can be visualized and quantified by spectrophotometry. There was no difference in the amount of EBD detected in the CNS of Mx1.Cre⁺α4-integrin^{fl/fl} mice and C57BL/6 control mice treated in vivo with poly I:C (FIG. 4A). A difference was not observed in the absolute number of inflammatory infiltrates in the spinal cords between animals of both mouse strains in whom active EAE had been induced in the absence of presence of poly I:C (FIG. 4 B-E). The anatomical locations of BBB compromise of Mx1.Cre⁺α4-integrin^{fl/fl} mice and C57BL/6 control mice treated vivo with poly I:C as indicated by EBD extravasation differed between mouse strains in the brains (FIG. 4 F&G; Table 1), and in the spinal cords (FIG. 4 H&I; Table 1).

Evaluation of BBB Permeability.

Mice were injected intravenously (i.v.) with 200 μL of 3% (weight/volume) Evans Blue dye and perfused with 4% paraformaldehyde after 3 hours. Brains and spinal cords were fixed in 4% paraformaldehyde and photographed with a dissecting microscope. For quantification of Evans Blue dye, tissues were dried at 56° C. overnight, then incubated with 8 mL/g N N-dimethylformamide at 56° C. for 48 hours. Evans Blue dye is soluble in N N-dimethylformamide, therefore we prepared exponential dilutions for a standard curve and measured absorbance with spectrophotometer at 650 nm (Xu et al., Investigative ophthalmology & visual science 2001; 42(3): 789-94; Ibla and Khoury, Methods in molecular biology (Clifton, N.J.) 2006; 341: 111-7).

Histology.

Brains were perfused and isolated as described above and fixed in 10% formalin. Brains were then coronally sectioned, embedded in Tissue-tek O.C.T. Compound, and snap frozen in liquid nitrogen. Six μm thick section were cut utilizing a freezing microtome and mounted on Fisher Brand Superfrost Plus glass slides. Samples were stained with hemotoxylin and eosin (H&R (Fisher Scientific, Pittsburgh, Pa.)) and prepared for light microscopy examination.

TABLE 1
TLR3 agonism through poly I:C treatment leads to differential anatomical
compromise of the blood-brain barrier demonstrated by Evans Blue Dye
(EBD) in Mx1.Crea⁺α4-integrin^{fl/fl} mice and C57BL/6 control mice.

| Genotype | Treatment | Tissue | Location of inflammation |
|---|---|---|---|
| Mx1.Cre^{+/+}α4- | Poly I:C | Brain | Ventral anterior cochlear nucleus |
| integrin^{fl/fl} | EAE | | Middle and inferior cerebellar |
| | | | peduncles |
| | | Spinal cord | Spinal cord white matter |
| Mx1.Cre^{+/+}α4- | EAE | Brain | Optic tract |
| integrin^{fl/fl} | | | Crus cerebri and pons |
| | | | Inferior cerebellar peduncle |
| | | Spinal cord | Spinal cord white matter |
| C57BL/6 | Poly I:C | Brain | Corticospinal tracts |
| | EAE | | Sensory trigeminal tract |
| | | | Spinal trigeminal tract |
| | | Spinal cord | Upper cervical spinal cord |
| | | | Spinal cord white matter |
| C57BL/6 | EAE | Brain | Optic tracts |
| | | | Corticospinal tracts |
| | | | Spinal trigeminal tract |
| | | Spinal cord | Spinal cord white matter |

### Example 9: In Vivo TLR3 Agonism Through Systemic Poly I:C Promotes Divers Integrin Usage in CNS-Infiltrating Leukocytes in the Setting of Relative α4-Integrin Deficiency

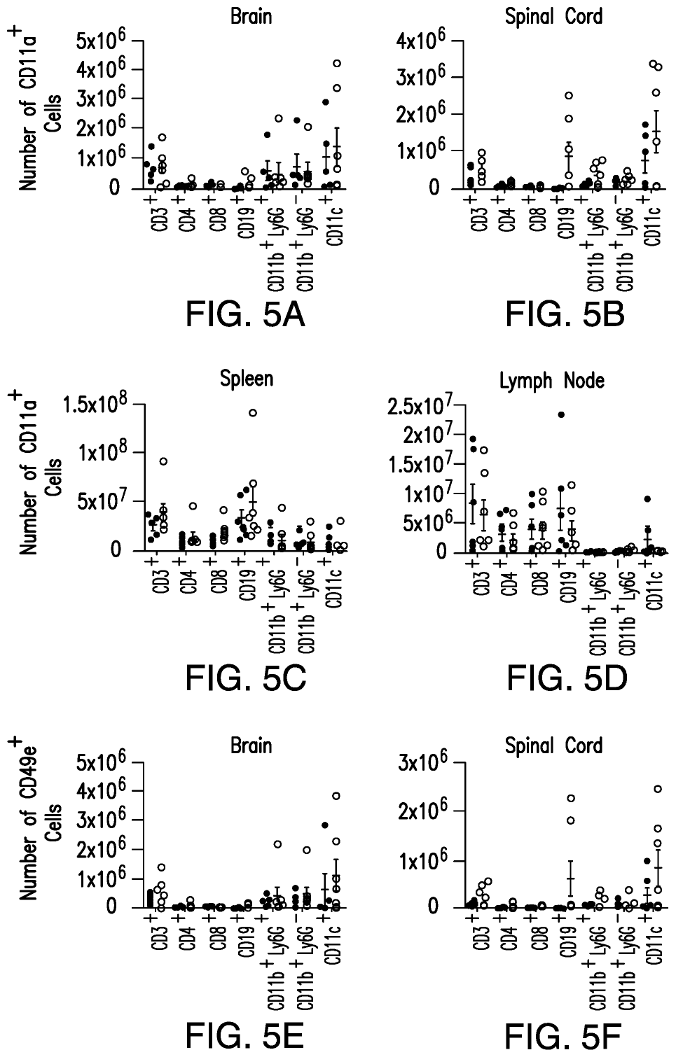

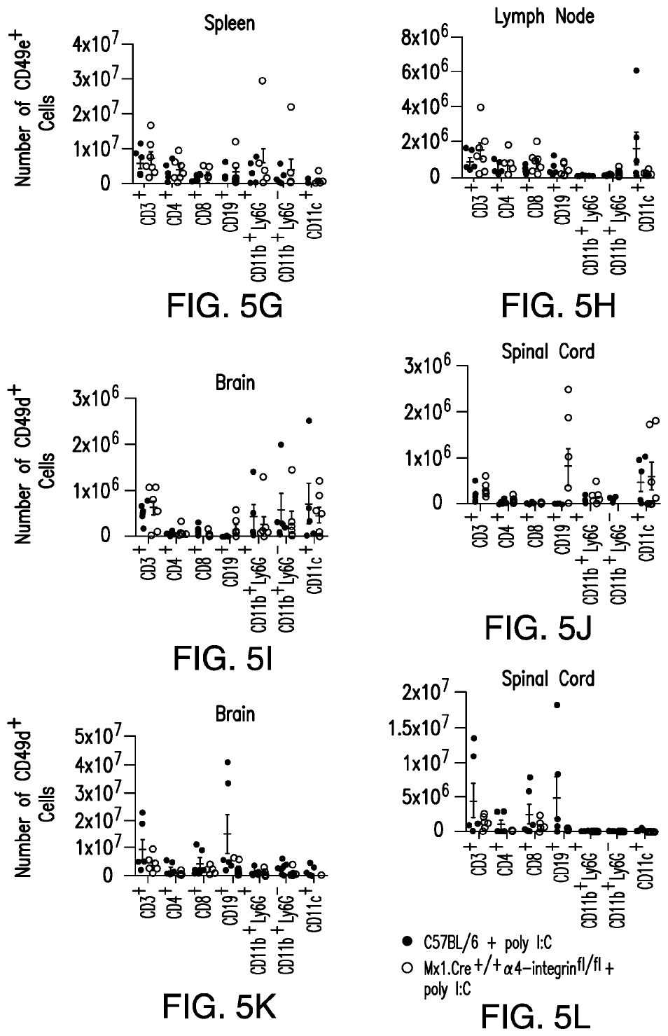

The experiments described herein indicated that TLR3 agonism through poly I:C re-establishes clinical and cellular immune competence in the CNS in the setting of relative α4-integrin deficiency. To determine the integrin usage required for leukocytes migration into the brain and spinal cords, the expression of Lymphocyte-function associated antigen-1 (LFA-1; β2-integrin; CD11a), (FIG. 5 A-D), α5-integrin (CD49e) (FIG. 5 E-H), and α4-integrin (CD49d) (FIG. 5 I-L) was assessed on different lymphocyte and myeloid cell subsets in Mx1.Cre⁺α4-integrin^{fl/fl} mice and C57BL/6 control mice actively induced for EAE on day 15. The number leukocytes subsets expressing CD11a, CD49e, and CD49d in all compartments was similar between mouse strains (FIG. 5 A-L). These results indicate that TLR3 agonism with poly I:C permits full access of α4-integrin-expressing leukocytes to the brain, even in the setting of relative α4-integrin deficiency. A high prevalence of activated T cells in the CNS (FIG. 3G), and BBB-compromise (FIG. 4) may be contributing factors underlying this observation.

### Example 10: TLR3 Agonism Through Systemic Poly I:C Administration Differentially Impacts Cytokine Expression in a Compartment-Specific Manner in the Setting of Relative α4-Integrin Deficiency

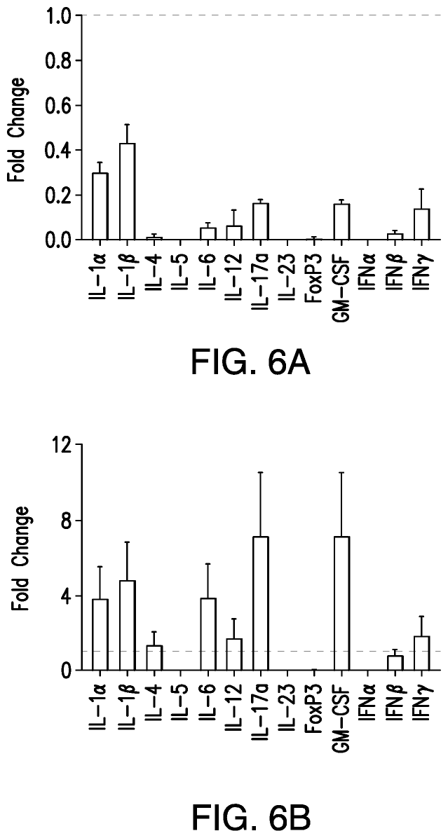

Engagement of TLR3 results in the transcription and cellular expression of type I IFN. To confirm that systemic administration of poly I:C induces expression of type I IFN within the CNS, and to confirm reports by Field et al (Field et al., Brain, behavior, and immunity 2010; 24(6): 996-1007), quantitative real-time PCR was performed for numerous cytokines in the brain and spinal cord of Mx1.Cre⁺α4-integrin^{fl/fl} mice and C57BL/6 control mice actively induced for EAE on day 15. In the brain, a decrease in transcription of IFNβ (FIG. 6A) was observed. Also, the transcription of numerous interleukins, GM-CSF, and the transcription factor FoxP3 was diminished in Mx1.Cre⁺α4-integrin^{fl/fl} mice. In spinal cord, the transcription of IFNβ in Mx1.Cre⁺α4-integrin^{fl/fl} mice was indistinguishable from that in C57BL/6 control mice, indicating that systemic administration of poly I:C induces type I IFN expression in this compartment (FIG. 6A). Several pro-inflammatory cytokines, including IL-1α, IL-1β, IL-6, IL-12, IL17a, and IFNγ were also substantially upregulated (FIG. 6A). The differential expression of cytokines in brain and spinal cord is likely explained by the different inflammatory environment in both organs. EAE in C57BL/6 mice is predominantly a spinal cord disease (Racke, CurrProtocNeurosci 2001; Chapter 9: Unit9), and the results described herein show that poly I:C disruption of the BBB affects different anatomical sites in Mx1.Cre⁺α4-integrin^{fl/fl} mice and C57BL/6 control mice (Table 1). Also, type I IFN is part of cytokine networks that involve both Th1 cell (Manca et al., Journal of interferon & cytokine research: the official journal of the International Society for Interferon and Cytokine Research 2005; 25(11): 694-701) and Th17 cell (Henry et al., J Immunol 2010; 184(7): 3755-67) development and function.

RNA Isolation and Quantitative Real-Time PCR.

TRI Reagent® Solution was utilized for RNA extraction of freshly isolated tissues of mice sacrificed on day 15 post immunization. Mice were overdosed with 400 mg/kg tribromomethanol and transcardially perfused with 20 mL ice cold PBS. Spleen, brain and spinal cord tissues were placed in 10-20 volumes of TRI Reagent solution after dissection. Tissues were homogenized in a glass homogenizer, transferred into a new tube and allowed to rest for 5 minutes at RT. 200 μl chlorophorm was added to each sample, mixed vigorously for 15 seconds, and centrifuged at 12000 g for 15 minutes at 4° C. After centrifugation, the upper aqueous phase was transferred into a new tube. An equal amount of isopropanol was added and incubated on ice for 15 minutes. Samples were centrifuged at 12000 g for 15 minutes at 4° C. Supernatant was decanted and the pellet was washed twice with 75% ethanol, dried and resuspended with 100 μl DEPC (diethylpyrocarbonate)-treated H₂O. RNA concentration was measured with a NanoDrop (Thermo Scientific NanoDrop™ 1000 Spectrophotometer). Taqman gene expression assays and the Step One Plus (Applied Biosystems. Foster City, Calif.) were utilized to detect IFNγ, IL-17a, IL-12a, Csf2 (GM-CSF), IL-23a, IL-6. Fold change in expression relative to untreated group was determined using the ddCt algorithm method described by the seller. The dCt was normalized to the housekeeping gene GAPDH.

What is claimed is:

1. A method of treating multiple sclerosis in a subject, the method comprising:
(a) identifying a subject in need of treatment; and
(b) administering to the subject a therapeutically effective amount of natalizumab and a toll-like receptor 3 (TLR3) agonist.

2. The method of claim 1, wherein the TLR3 agonist is rintatolimod, poly-L-lysine (Poly ICLC), or polyinosinic-polycytidylic acid (Poly (I:C)).

3. The method of claim 1, wherein the multiple sclerosis is relapsing remitting, secondary progressive, or primary progressive multiple sclerosis.

4. The method of claim 2, wherein the administration of the natalizumab is before, during or after the administration of the Poly (I:C).

5. The method of claim 1, wherein the subject has been identified as being at risk for progressive multifocal leukoencephalopathy prior to the administering step of the TLR3 agonist.

6. The method of claim 1, further comprising monitoring the subject for indicators of progressive multifocal leukoencephalopathy.

7. The method of claim 1, wherein the subject is a human.

8. The method of claim 1, wherein the natalizumab is administered intravenously.

9. The method of claim 2, wherein the Poly (I:C) is administered intravenously or intranasally.

10. The method of claim 4, wherein natalizumab and Poly (I:C) are administered to the patient over a series of treatments.

11. The method of claim 1, wherein the patient is undergoing natalizumab treatment.

12. The method of claim 1, wherein the patient has been diagnosed with progressive multifocal encephalopathy (PML).

13. A method of treating a patient at risk of having progressive multifocal leukoencephalopathy (PML), the method comprising: administering to a patient a therapeutically effective amount of a toll-like receptor 3 (TLR3) agonist.

14. The method of claim 13, wherein the patient has undergone therapy with natalizumab.

15. The method of claim 13, wherein the patient has been diagnosed with multiple sclerosis.

16. The method of claim 15, wherein the multiple sclerosis is relapsing remitting, secondary progressive, or primary progressive sclerosis.

17. The method of claim 13, wherein the patient has been diagnosed with Crohn's disease.

18. The method of claim 13, wherein the TLR3 agonist is rintatolimod, poly-L-lysine (Poly ICLC), or polyinosinic-polycytidylic acid (Poly (I:C)).

19. The method of claim 18, wherein the Poly (I:C) is administered intravenously or intranasally.

20. A method of activating T cells in a subject, the method comprising: administering a therapeutically effective amount of a composition comprising polyinosinic-polycytidylic acid (Poly (I:C)) to a subject having or suspected of having a reduced number of T cells; wherein the subject has previously undergone treatment with natalizumab or is currently undergoing treatment with natalizumab; and wherein the number of T cells is increased after administration of the Poly (I:C).

21. The method of claim 20, wherein the patient has been diagnosed with multiple sclerosis.

22. The method of claim 20, wherein the multiple sclerosis is relapsing remitting, secondary progressive, primary progressive or chronic progressive multiple sclerosis.

23. The method of claim 20, wherein the patient has been diagnosed with Crohn's disease.

24. A method of preventing progressive multifocal leukoenchephalopathy (PML) in a subject with multiple sclerosis or Crohn's disease, the method comprising:
(a) identifying a subject in need of treatment; and
(b) administering to the subject a therapeutically effective amount of a toll-like receptor 3 agonist before, during or after administration of natalizumab, in an amount sufficient to prevent PML.
